(19) United States
(12) Patent Application Publication
(10) Pub. No.: US 2026/0365634 A1
(43) Pub. Date: Nov. 19, 2026

(54) HERBICIDAL PYRAZOLE COMPOUNDS

(71) Applicant: SYNGENTA CROP PROTECTION AG, Basel (CH)

(72) Inventors: Suzanna Jane DALE, Bracknell, Berkshire (GB); Philip Michael ELVES, Bracknell, Berkshire (GB); James Alan MORRIS, Bracknell, Berkshire (GB); Vikas SIKERVAR, Bracknell, Berkshire (GB)

(73) Assignee: SYNGENTA CROP PROTECTION AG, Basel (CH)

(21) Appl. No.: 19/107,250

(22) PCT Filed: Aug. 25, 2023
(86) PCT No.: PCT/EP2023/073328

§ 371 Date: Feb. 27, 2025

(30) Foreign Application Priority Data
Sep. 1, 2022 (IN) 202211050045

(51) Int. Cl. C07D 403/06; A01N 43/56; A01N 43/653; A01N 43/80; A01P 13/02; C07D 401/06; C07D 403/14; C07D 405/14; C07D 413/14

(52) U.S. Cl. CPC C07D 403/06; A01N 43/56; A01N 43/653; A01N 43/80; A01P 13/02; C07D 401/06; C07D 403/14; C07D 405/14; C07D 413/14

(57) ABSTRACT

The present invention relates to compounds of Formula (I), or an agronomically acceptable salt of said compounds wherein A, Q, R¹, R², R³ and m are as defined herein. The invention further relates to herbicidal compositions which comprise a compound of Formula (I) and to the use of compounds of Formula (I) for controlling weeds, in particular in crops of useful plants.

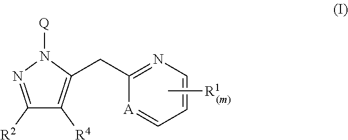

The present invention relates to herbicidal compounds, to processes for their preparation, to herbicidal compositions which comprise the compounds, and to their use for controlling weeds, in particular in crops of useful plants, or for inhibiting plant growth.

Herbicidal pyrazole compounds are known, for example, from CN105037342, WO2022/013293, WO2022/101270 and WO2023/099354. The present invention relates to alternative herbicidal pyrazole compounds. Thus, according to the present invention there is provided a compound of Formula (I):

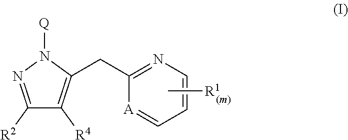

or an agronomically acceptable salt thereof,
wherein
A is CR¹¹ or N;
Q is selected from the group consisting of hydrogen, C₁-C₈ alkyl, C₂-C₈ alkenyl, C₂-C₈ alkynyl, C₁-C₈ haloalkyl, C₂-C₈ haloalkenyl, C₂-C₈ haloalkynyl, C₁-C₄alkoxy-C₁-C₃alkyl-, C₁-C₄haloalkoxy-C₁-C₃alkyl-, C₁-C₄alkoxy-C₁-C₃haloalkyl-, C₁-C₈cyanoalkyl- (e.g —CH₂CH₂CN), C₃-C₆ cycloalkyl (optionally substituted by CN, fluoro or chloro), —S(O)ₚR⁴ and —(CH₂)ₙR¹⁰;
R¹ is independently selected from the group consisting of halogen, —CN, C₁-C₂alkyl, C₁-C₂haloalkyl, C₃-C₆cycloalkyl, C₁-C₂alkoxy- and C₁-C₂haloalkoxy-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)ₚC₁-C₄alkyl, —C(R⁶)═NOR⁷ and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR₃R⁹;
R⁴ is selected from the group consisting of C₁-C₄alkyl, C₁-C₄haloalkyl and C₃-C₆cycloalkyl;
R⁵ is selected from the group consisting of hydrogen, methyl, ethyl, —(CH₂)-cyclopropyl, C₂-C₃ alkenyl, C₂-C₃ alkynyl and cyclopropyl;
R⁶ is selected from the group consisting of hydrogen, methyl and ethyl;
R⁷ is methyl or ethyl;
R³ is hydrogen or C₁-C₄alkyl;
R⁹ is hydrogen or C₁-C₄alkyl;
R¹⁰ is selected from the group consisting of —OH, —C(H)O, —C(O)R⁴, —S(O)ₚR⁴, —C(H)(OH)—CH₂OH, —C(R⁶)═NOR⁵, —NR¹²R¹³, C₃-C₆ cycloalkyl (optionally substituted by CN, fluoro or hydroxy), a C₃-C₆ saturated heterocycle comprising one or more oxygen atoms (e.g tetrahydropyran and tetrahydrofuran), and a 5-membered heteroaryl (e.g pyrazolyl) which comprises from 1 to 3 heteroatoms each independently selected from the group consisting of oxygen, nitrogen and sulphur, wherein said heteroaryl is optionally substituted by one or two substituents independently selected from the group consisting of halogen, cyano, C₁-C₂ alkyl, C₁-C₂ haloalkyl, C₃-C₄cycloalkyl, C₁-C₂ alkoxy and C₁-C₄ haloalkoxy; R¹¹ is selected from the group consisting of hydrogen, fluoro, chloro and CN; R¹² and R¹³ are independently selected from the group consisting of C₁-C₄alkyl, C₃-C₄cycloalkyl, C₁-C₄alkyl-S(O)ₚ—, C₃-C₄cycloalkyl-S(O)ₚ—, C₁-C₄alkyl-C(O)— and C₃-C₄cycloalkyl-C(O)—;
m=0, 1 or 2;
n=1, 2 or 3; and
p=0, 1 or 2.

C₁-C₄alkyl- and C₁-C₆alkyl- includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine. The same correspondingly applies to halogen in the context of other definitions, such as haloalkyl.

C₁-C₈haloalkyl- includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl. C₁-C₄haloalkyl- and C₁-C₂haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄alkoxy and C₁-C₃alkoxy includes, for example, methoxy and ethoxy.

C₁-C₃haloalkoxy- and C₁-C₃haloalkoxy- include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₈alkenyl- includes, for example, —CH═CH₂ (vinyl) and —CH₂—CH═CH₂ (allyl).

C₂-C₈alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention, A is CR¹¹ and R¹¹ is selected from the group consisting of hydrogen, fluoro, chloro and CN, preferably fluoro or hydrogen.

In one embodiment of the present invention, A is N. In another embodiment of the present invention m is 1. Thus, in a preferred embodiment of the present invention there is provided a compound of Formula (Ia)

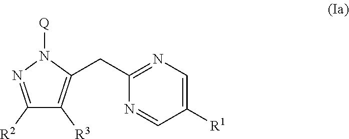

wherein Q, R¹, R² and R³ are as defined in claim 1 above.

In one embodiment of the present invention there is provided a compound of Formula (I) or Formula (Ia), wherein R¹ is chloro.

In one embodiment of the present invention there is provided a compound of Formula (I) or Formula (Ia), wherein R² is halogen (e.g Cl or Br) or C₁-C₄haloalkyl (e.g —CF₃, —CHF₂, —CF₂Cl).

In one embodiment of the present invention there is provided a compound of Formula (I) or Formula (Ia), wherein, R³ is hydrogen or halogen (e.g Cl).

In a preferred embodiment of the present invention there is provided a compound of Formula (I) or Formula (Ia), wherein Q is selected from the group consisting of hydrogen, C₁-C₈ alkyl (e.g methyl, —CH₂CH(CH₃)₂, n-butyl, n-pentyl), C₂-C₈alkenyl (e.g —CH₂CH₂CH═CH₂), C₁-C₈ haloalkyl (e.g —CH₂CH₂CHF₂, —CH₂CH₂CH₂CHF₂, —CH₂CH₂CF₃, —CH₂CH₂CH₂CF₃), C₁-C₄alkoxy-C₁-C₃alkyl- (e.g —CH₂OCH₃, —CH₂CH₂OCH₃), C₁-C₄haloalkoxy-C₁-C₃alkyl- (e.g —CH₂OCH₂CHF₂, —CH₂CH₂OCH₂CHF₂), —S(O)ₚR⁴ (e.g —SO₂CH₂CH₂CF₃, —S—CH₂CH₂CF₃) and —(CH₂)ₙR¹⁰ (e.g —CH₂CH₂CH₂OH, —CH₂CH₂C(H)(OH)CH₂OH, —CH₂CH₂C(H)O)—OH, —CH₂CH₂C(H)═N—OH, —CH₂CH₂C(H)═N—O—CH₃ and —CH₂CH₂C(H)═N—O—CH₂-cPr).

In one embodiment of the present invention there is provided a compound of Formula (I) or Formula (Ia), wherein Q is —(CH₂)ₙR¹⁰, n is 1, 2 or 3 and R¹⁰ is selected from the group consisting of —OH (e.g —CH₂CH₂CH₂OH), —C(H)(OH)—CH₂OH (e.g —CH₂CH₂C(H)(OH)CH₂OH), —C(H)═NOR⁵ (e.g —CH₂CH₂C(H)═N—OH, —CH₂CH₂C(H)═N—O—CH₃, —CH₂CH₂C(H)═N—O—CH₂-cPr), and —NR¹²R¹³ (e.g —CH₂CH₂CH₂N(CH₃)SO₂CH₃.

In a preferred embodiment of the present invention, there is provided a compound of Formula (I) or Formula (Ia), wherein Q is C₁-C₈ alkyl (e.g methyl, —CH₂CH(CH₃)₂, n-butyl, n-pentyl).

In another preferred embodiment of the present invention, there is provided a compound of Formula (I) or Formula (Ia), wherein Q is C₁-C₈ haloalkyl (e.g —CH₂CH₂CHF₂, —CH₂CH₂CHF₂, —CH₂CH₂CF₃, —CH₂CH₂CH₂CF₃).

In another preferred embodiment of the present invention, there is provided a compound of Formula (I) or Formula (Ia), wherein Q is C₁-C₄haloalkoxy-C₁-C₃alkyl-e.g —CH₂OCH₂CHF₂, —CH₂CH₂CH₂OCH₂CHF₂).

The present invention further relates to a compound of Formula (II-a):

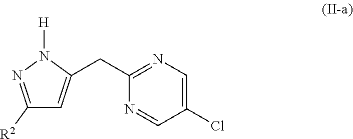

wherein R² is CF₂H or CF₂Cl.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70 °C) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester,4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate,6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, tetrahydrofuran-2-ylmethyl (2R)-2-[(4-amino-3,5-dichloro-6-fluoro-2-pyridyl)oxy]propanoate, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate and 6-amino-5-chloro-2-(4-chloro-2-fluoro-3-methoxy-phenyl)pyrimidine-4-carboxylic acid.

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much-improved selectivity compared to know, structurally similar compounds. Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. The compounds of the present invention may also be used in conjunction with plants disclosed in WO2020/236790.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK@(Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B@(cotton), Bollgard@(cotton), NewLeaf® (potatoes), NatureGardO and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

### Processes for Preparation of Compounds of Formula (I)

Processes for preparation of compounds, e.g. a compound of formula (I) (which optionally can be an agrochemically acceptable salt thereof), are now described, and form further aspects of the present invention.

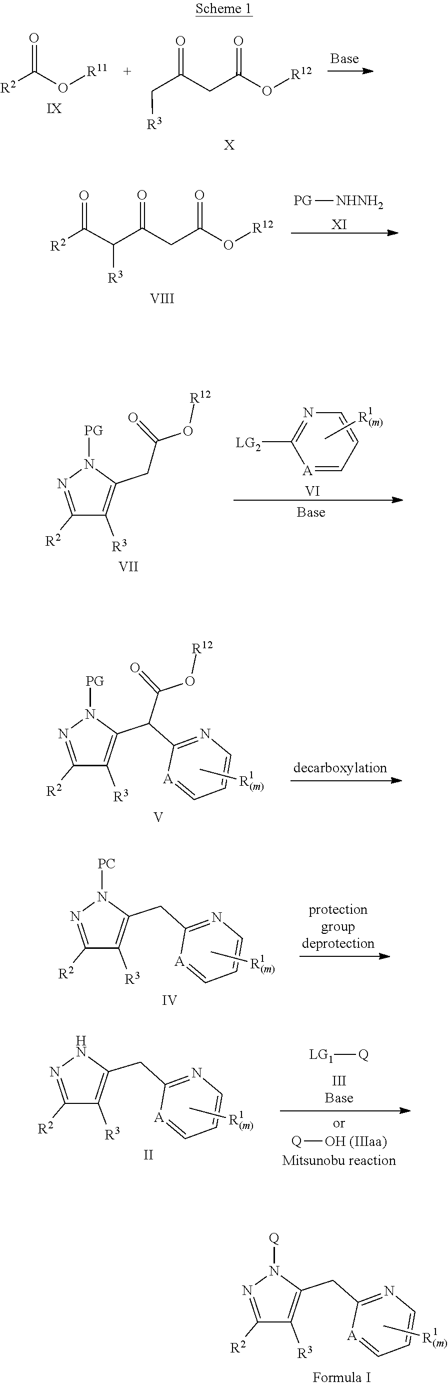

In scheme 1 compounds of formula I can be prepared by reacting compounds of formula II, with reagents of the formula III, wherein LG₁ is a halogen, preferably iodine, bromine or chlorine (or a pseudo-halogen leaving group, such as a (halo)alkyl or phenyl sulfonate ester, e.g. triflate), in the presence of a base, such as sodium hydride or an alkali earth metal hydride, carbonate (e.g. sodium carbonate, potassium carbonate or cesium carbonate) or hydroxide, optionally in the presence of potassium iodide in an inert solvent such as tetrahydrofuran, dioxane, water, N,N-dimethylformamide DMF, N,N-dimethylacetamide, sulfolane or acetonitrile and the like, at temperatures between 0 and 120 °C, by procedures well known to those skilled in the art. Alternatively compounds of formula I can be prepared by reacting compounds of formula II with compounds of formula Illaa under Mitsunobu reaction conditions. Such reactions are well known to those skilled in the art and can be carried out in the presence of a suitable phosphine reagent such as triphenylphosphine or tributylphosphine and similar others and in the presence of an azodicarboxylate reagent such as diisopropyl azodicarboxylate or diethyl azodicarboxylate. The reaction is generally carried out in the presence of a solvent such as dichloromethane, tetrahydrofuran, 2-methyltetrahydrofuran, toluene, and similar others and at temperature preferably in the range of −10 °C to the boiling point of solvent.

Compounds of formula II can be prepared by protecting group deprotection reaction from compounds of formula IV, wherein PG is a N-protecting group for example acetyl, trimethylsilylethoxymethyl (SEM), tert-butyloxycarbonyl, benzyl, p-methoxybenzyl (PMB) amongst others amino protecting groups. Such reactions are well known to those skilled in the art and can be carried out for example using base catalyzed such as using sodum hydroxide for the deprotection of acetyl group or acid catalyzed such as hydrochloric acid or 2,2,2-trifluoroacetic acid for the deprotection of trimethylsilylethoxymethyl (SEM), tert-butyloxycarbonyl or p-methoxybenzyl (PMB) group.

Compounds of formula IV can be prepared from compounds of formula V, wherein R¹² is C₁-C₄alkyl or phenyl via decarboxylation reaction. The reaction can be carried out using base such as alkaline earth metal hydroxide or alkali metal hydroxide like sodium hydroxide or in the presence of acid such as aquous hydrochloric acid, sulfuric acid amongst others. The reaction is generally carried out in the presence of a solvent such as water, ethanol, methanol, tetrahydrofuran or dioxane or combination of two or more solvent and at temperature in the range of room temperature to boiling point of solvent.

Compounds of formula V, wherein R¹² is C₁-C₄alkyl or phenyl can be prepared by reacting compounds of formula VII, with reagents of the formula VI, wherein LG₂ is a halogen, preferably iodine, bromine or chlorine (or a pseudo-halogen leaving group, such as a (halo)alkyl or phenyl sulfonate ester, e.g. triflate), in the presence of a base, such as sodium tert-butoxide, sodium hydride or an alkali earth metal hydride, carbonate (e.g. sodium carbonate, potassium carbonate or cesium carbonate) or hydroxide, or phosphate such as potassium phosphate optionally in the presence of potassium iodide in an inert solvent such as tetrahydrofuran, dioxane, water, N,N-dimethylformamide DMF, N,N-dimethylacetamide, dimethyl sulfoxide, sulfolane or acetonitrile and the like, at temperatures between 0 and 120 °C, by procedures well known to those skilled in the art.

Compounds of formula VII can be prepared by the condensation reaction of compounds of formula VIII with compounds of formula XI (or its hydrochloric acid salt or trifluoroacetic acid salt), wherein PG is a N-protecting group for example acetyl, trimethylsilylethoxymethyl (SEM), tert-butyloxycarbonyl, benzyl, p-methoxybenzyl (PMB) amongst others amino protecting groups. Such reactions are well known in the literature and can be carried out optionally in the presence of an acid catalyst such as acetic acid.

Compounds of formula VIII can be prepared by reacting compounds of formula IX, wherein R¹¹ is C₁-C₄alkyl or phenyl with compounds of formula X in the presence of a base. Such reactions are known by the name of Claisen condensation reaction and well known to those skilled in the art. Reaction can be carried out using base such as lithium diisopropylamide, lithium tetramethylpiperidide, sodium ethoxide, sodium hydride amongst other bases in the presence of solvent such as tetrahydrofuran, ethanol, methanol and at temperature in the range of −80 °C to boiling point of solvent.

The following non-limiting examples provide specific synthesis methods for representative compounds of the present invention, as referred to in the Table below.

### Example 1: Preparation of 5-chloro-2-[[2-(4,4-difluorobutyl)-5-(trifluoromethyl)pyrazol-3-yl]methyl]pyrimidine (1.005)

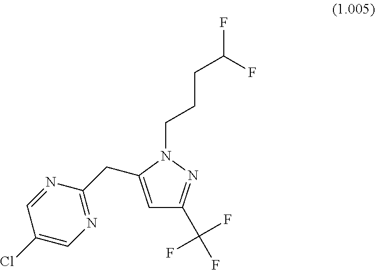 [SMILES: FC(F)CCCn1nc(C(F)(F)F)cc1Cc1ncc(Cl)cn1]

### Step 1: Preparation of ethyl 6,6,6-trifluoro-3,5-dioxo-hexanoate (I1)

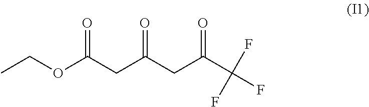

To an ice-cooled solution of diisopropylamine (22 mL, 157 mmol) in THF (110 mL) in a 250 mL round bottomed flask was added n-butyllithium (2.5 M in hexanes, 65 mL, 160 mmol) over approximately 30 minutes. The mixture was stirred for 30 minutes, treated dropwise with ethyl acetoacetate (6.8 mL, 54 mmol) and was stirred for a further 45 minutes at 0 °C. The reaction mixture was then cooled to −78 °C, treated dropwise with ethyl trifluoroacetate (8.3 mL, 70 mmol) and stirred for 3 hours. The mixture was quenched with hydrochloric acid, diluted with water and extracted with ethyl acetate. The combined organics were dried and concentrated to provide ethyl 6,6,6-trifluoro-3,5-dioxo-hexanoate I1 as an oil which was used as such in the next step.

### Step 2: Preparation of ethyl 2-[2-[(4-methoxyphenyl)methyl]-5-(trifluoromethyl)pyrazol-3-yl]acetate (I2)

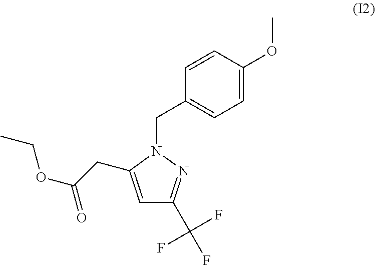 [SMILES: CCOC(=O)Cc1cc(C(F)(F)F)nn1Cc1ccc(OC)cc1]

To a solution of the ethyl 6,6,6-trifluoro-3,5-dioxo-hexanoate I1 (prepared in step 1) in acetic acid (90 mL) in a 100 mL flask was added (4-methoxybenzyl)hydrazine hydrochloride (10.56 g, 54.29 mmol) and the resulting mixture was allowed to stir for 1.5 hours at room temperature. The mixture was diluted with tert-butylmethyl ether and washed with water and brine. The organics were concentrated and subjected to column chromatography on silica gel using 0-30% ethyl acetate in cyclohexane to give ethyl 2-[2-[(4-methoxyphenyl)methyl]-5-(trifluoromethyl)pyrazol-3-yl]acetate I2 as a yellow oil (11.97 g, 59% yield). ¹H NMR (400 MHz, CDCl₃) δ=7.10-7.04 (m, 2H), 6.88-6.83 (m, 2H), 6.50 (s, 1H), 5.35 (s, 2H), 4.12 (q, 2H), 3.79 (s, 3H), 3.54 (s, 2H), 1.24 (t, 3H)

### Step 3: Preparation of ethyl 2-(5-chloropyrimidin-2-yl)-2-[2-[(4-methoxyphenyl)methyl]-5-(trifluoromethyl)pyrazol-3-yl]acetate (I3)

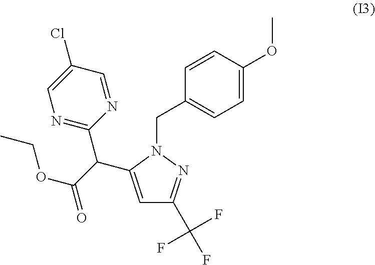 [SMILES: CCOC(=O)C(c1ncc(Cl)cn1)c1cc(C(F)(F)F)nn1Cc1ccc(OC)cc1]

To a solution of ethyl 2-[2-[(4-methoxyphenyl)methyl]-5-(trifluoromethyl)pyrazol-3-yl]acetate I2 (4.22 g, 11.71 mmol) in dimethyl sulfoxide (40 mL) in a 250 mL flask was added 2,5-dichloropyrimidine (3.71 g, 24.9 mmol) and potassium phosphate tribasic (7.86 g, 36.3 mmol) and the reaction mixture was stirred at 60 °C for 3.5 hours. The mixture was allowed to stand at room temperature overnight and then was stirred at 60 °C for an additional hour. The mixture was cooled to room temperature, diluted with water and extracted with tert-butylmethyl ether. The combined organics were concentrated and subjected to column chromatography on silica gel using 0-40% ethyl acetate in cyclohexane to give ethyl 2-(5-chloropyrimidin-2-yl)-2-[2-[(4-methoxyphenyl)methyl]-5-(trifluoromethyl)pyrazol-3-yl]acetate I3 as a yellow oil (5.19 g, 73% yield). ¹H NMR (400 MHz, CDCl₃) δ=8.55 (s, 2H), 6.98-6.92 (m, 2H), 6.79-6.74 (m, 2H), 6.69 (s, 1H), 5.45-5.31 (m, 3H), 4.20-4.11 (m, 2H), 3.77 (s, 3H), 1.21-1.16 (m, 3H)

### Step 4: Preparation of 5-chloro-2-[[2-[(4-methoxyphenyl)methyl]-5-(trifluoromethyl)pyrazol-3-yl]methyl]pyrimidine (I4)

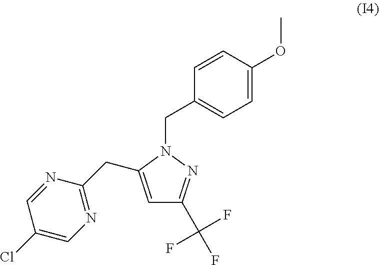 [SMILES: COc1ccc(Cn2nc(C(F)(F)F)cc2Cc2ncc(Cl)cn2)cc1]

To a solution of ethyl 2-(5-chloropyrimidin-2-yl)-2-[2-[(4-methoxyphenyl)methyl]-5-(trifluoromethyl)pyrazol-3-yl]acetate I3 (5.19 g, 8.56 mmol) in methanol (40 mL) in a 250 mL flask was added 2 M aqueous sodium hydroxide (40 mL) and the reaction mixture was stirred at 60 °C for 1.5 hours. The mixture was cooled to room temperature and concentrated to half volume. The residues were then acidified with hydrochloric acid and extracted with ethyl acetate. The combined organics were dried and concentrated to give 5-chloro-2-[[2-[(4-methoxyphenyl)methyl]-5-(trifluoromethyl)pyrazol-3-yl]methyl]pyrimidine I4 as an orange oil (3.92 g, quantitative yield). ¹H NMR (400 MHz, CDCl₃) δ=8.55 (s, 2H), 7.05-6.95 (m, 2H), 6.84-6.75 (m, 2H), 6.47 (s, 1H), 5.40 (s, 2H), 4.22 (s, 2H), 3.78 (s, 3H)

### Step 5: Preparation of 5-chloro-2-[[3-(trifluoromethyl)-1H-pyrazol-5-yl]methyl]pyrimidine (I5)

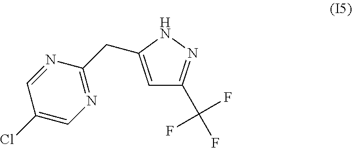 [SMILES: FC(F)(F)c1cc(Cc2ncc(Cl)cn2)[nH]n1]

A solution of 5-chloro-2-[[2-[(4-methoxyphenyl)methyl]-5-(trifluoromethyl)pyrazol-3-yl]methyl]pyrimidine I4 (3.92 g, 8.56 mmol) in 2,2,2-trifluoroacetic acid (14 mL) in a 100 mL flask was stirred at 90 °C for one hour. The mixture was cooled to room temperature, slowly transferred into an aqueous sodium bicarbonate solution and then extracted with ethyl acetate. The combined organics were concentrated and subjected to column chromatography on silica gel using 0-60% ethyl acetate in cyclohexane to give 5-chloro-2-[[3-(trifluoromethyl)-1H-pyrazol-5-yl]methyl]pyrimidine I5 as an off-white solid (2.365 g, 98% yield). ¹H NMR (400 MHz, CDCl₃) δ=8.70 (s, 2H), 6.49 (s, 1H), 4.41 (s, 2H)

### Step 6: Preparation of 5-chloro-2-[[2-(4,4-difluorobutyl)-5-(trifluoromethyl)pyrazol-3-yl]methyl]pyrimidine (1.005)

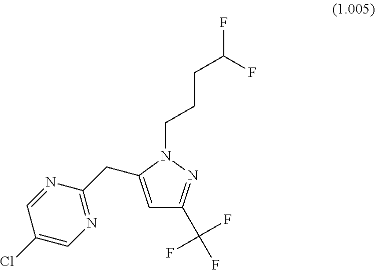 [SMILES: FC(F)CCCn1nc(C(F)(F)F)cc1Cc1ncc(Cl)cn1]

To a 25 mL flask was added 5-chloro-2-[[3-(trifluoromethyl)-1H-pyrazol-5-yl]methyl]pyrimidine I5 (98 mg, 0.336 mmol) and potassium carbonate (129 mg, 0.933 mmol). The mixture was treated with a solution of 4-bromo-1,1-difluorobutane (90 mg, 0.520 mmol) in acetonitrile (1.5 mL) and was stirred at 70 °C and stirred for 1.5 hours. On completion, the mixture was allowed to cool before being diluted with water and extracted with ethyl acetate. The combined organics were concentrated and subjected prep-HPLC chromatography to give 5-chloro-2-[[2-(4,4-difluorobutyl)-5-(trifluoromethyl)pyrazol-3-yl]methyl]pyrimidine 1.005 as a colourless oil (27 mg, 22% yield). ¹H NMR (400 MHz, CDCl3) δ=8.65 (s, 2H), 6.43 (s, 1H), 5.83 (tt, 1H), 4.32 (s, 2H), 4.26 (t, 2H), 2.10-1.96 (m, 2H), 1.94-1.78 (m, 2H).

### Example 2: Preparation of 2-[[2-butyl-5-[chloro(difluoro)methyl]pyrazol-3-yl]methyl]-5-chloro-pyrimidine (1.020)

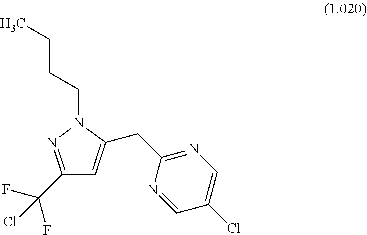 [SMILES: CCCCn1nc(C(F)(F)Cl)cc1Cc1ncc(Cl)cn1]

### Step 1: Preparation of ethyl 6-chloro-6,6-difluoro-3,5-dioxo-hexanoate (I6)

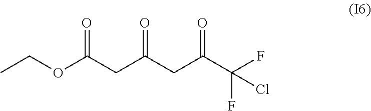

An ice-cooled solution of THF (80 mL) and lithium diisopropylamide solution (2.0 M in THF/heptane/ethylbenzene, 70 mL, 140 mmol) was treated dropwise with ethyl acetoacetate (5.10 mL, 40.3 mmol) and the resulting mixture was allowed to stir for 30 minutes at 0 °C. The reaction mixture was then cooled to −78 °C, treated dropwise with ethyl chlorodifluoroacetate (6.60 mL, 52.1 mmol) and stirred for an additional 3.5 hours. The reaction mixture was allowed to warm to room temperature over 30 minutes before being quenched with 1 M hydrochloric acid (200 mL) and extracted with ethyl acetate (2×80 mL). The combined organics were dried and concentrated to provide ethyl 6-chloro-6,6-difluoro-3,5-dioxo-hexanoate (I6) as an oil which was used directly in the next step.

### Step 2: Preparation of ethyl 2-[5-[chloro(difluoro)methyl]-2-[(4-methoxyphenyl)methyl]pyrazol-3-yl]acetate (I7)

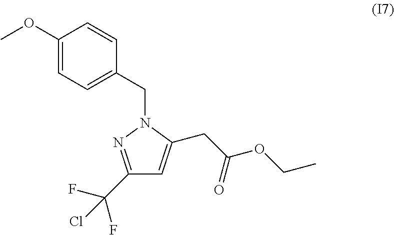 [SMILES: CCOC(=O)Cc1cc(C(F)(F)Cl)nn1Cc1ccc(OC)cc1]

To a solution of ethyl 6-chloro-6,6-difluoro-3,5-dioxo-hexanoate (I6) (prepared in step 1, example 2) in acetic acid (40 mL) in a 100 mL flask was added (4-methoxybenzyl)hydrazine hydrochloride (7.05 g, 36.3 mmol) and the resulting mixture was allowed to stir at room temperature for 15 hours. The reaction mixture was concentrated and subjected to column chromatography on silica gel using 0-30% ethyl acetate in cyclohexane to give ethyl 2-[5-[chloro(difluoro)methyl]-2-[(4-methoxyphenyl)methyl]pyrazol-3-yl]acetate I7 as a brown oil (7.87 g, 48% yield). ¹H NMR (400 MHz, chloroform) 6=7.10-7.04 (m, 2H), 6.89-6.82 (m, 2H), 6.50 (s, 1H), 5.35 (s, 2H), 4.12 (q, 2H), 3.79 (s, 3H), 3.53 (s, 2H), 1.24 (t, 3H)

### Step 3: Preparation of ethyl 2-[5-[chloro(difluoro)methyl]-2-[(4-methoxyphenyl)methyl]pyrazol-3-yl]-2-(5-chloropyrimidin-2-yl)acetate (I8)

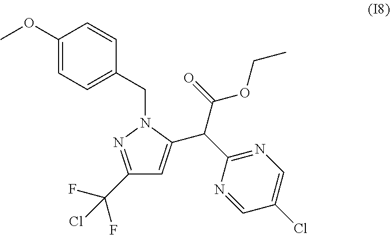 [SMILES: CCOC(=O)C(c1ncc(Cl)cn1)c1cc(C(F)(F)Cl)nn1Cc1ccc(OC)cc1]

To a solution of ethyl 2-[5-[chloro(difluoro)methyl]-2-[(4-methoxyphenyl)methyl]pyrazol-3-yl]acetate I7 (7.87 g, 21.9 mmol) in dimethyl sulfoxide (55 mL) in a 250 mL round bottomed flask was added 2,5-dichloropyrimidine (9.85 g, 66.1 mmol) and potassium phosphate tribasic (19.4 g, 89.5 mmol). The reaction mixture was warmed to 70 °C and was stirred for 3.5 hours. The reaction mixture was then cooled to room temperature before being diluted with 0.5 M hydrochloric acid (200 mL) and extracted with tert-butylmethyl ether (2×100 mL). The combined organic extracts were concentrated and subjected to column chromatography on silica gel using 0-30% ethyl acetate in cyclohexane to give ethyl 2-[5-[chloro(difluoro)methyl]-2-[(4-methoxyphenyl)methyl]pyrazol-3-yl]-2-(5-chloropyrimidin-2-yl)acetate 18 as a brown oil (6.53 g, 60%). ¹H NMR (400 MHz, chloroform) δ=8.55 (s, 2H), 6.99-6.92 (m, 2H), 6.79-6.75 (m, 2H), 6.68 (s, 1H), 5.45-5.33 (m, 3H), 4.20-4.09 (m, 2H), 3.77 (s, 3H), 1.19 (t, 3H).

### Step 4: Preparation of 5-chloro-2-[[5-[chloro(difluoro)methyl]-2-[(4-methoxyphenyl)methyl]pyrazol-3-yl]methyl]pyrimidine (I9)

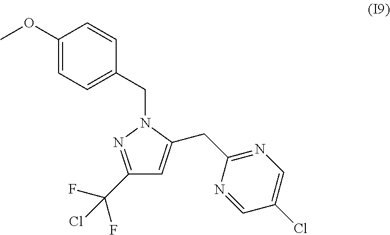 [SMILES: COc1ccc(Cn2nc(C(F)(F)Cl)cc2Cc2ncc(Cl)cn2)cc1]

A solution of ethyl 2-[5-[chloro(difluoro)methyl]-2-[(4-methoxyphenyl)methyl]pyrazol-3-yl]-2-(5-chloropyrimidin-2-yl)acetate 18 (6.52 g, 13.8 mmol) in methanol (70 mL) in a 250 mL round bottomed flask was treated with 2 M aqueous sodium hydroxide solution (70 mL, 140 mmol). The resulting mixture was warmed to 60 °C and was allowed to stir for 2 hours. The reaction mixture was cooled to room temperature before being diluted with ethyl acetate (100 mL) and neutralised with 2 M hydrochloric acid (100 mL). The organic layer was then separated, and the aqueous layer extracted with ethyl acetate (100 mL). The combined organic extracts were then concentrated and subjected to column chromatography on silica gel using 0-35% ethyl acetate in cyclohexane to give 5-chloro-2-[[5-[chloro(difluoro)methyl]-2-[(4-methoxyphenyl)methyl]pyrazol-3-yl]methyl]pyrimidine I9 as a beige oil (3.76 g). ¹H NMR (400 MHz, chloroform) δ=8.55 (s, 2H), 7.00 (d, 2H), 6.79 (d, 2H), 6.46 (s, 1H), 5.40 (s, 2H), 4.21 (s, 2H), 3.78 (s, 3H).

### Step 5: Preparation of 5-chloro-2-[[3-[chloro(difluoro)methyl]-1H-pyrazol-5-yl]methyl]pyrimidine (I10)

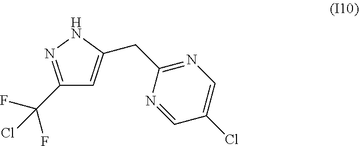 [SMILES: FC(F)(Cl)c1cc(Cc2ncc(Cl)cn2)[nH]n1]

A solution of 5-chloro-2-[[5-[chloro(difluoro)methyl]-2-[(4-methoxyphenyl)methyl]pyrazol-3-yl]methyl]pyrimidine I9 (3.756 g, 9.408 mmol) in 2,2,2-trifluoroacetic acid (15 mL) in a 100 mL round bottomed flask was warmed to 70 °C and was allowed to stir for 3 hours. The reaction mixture was allowed to cool to room temperature before being added to water (200 mL), causing a precipitate to form. The mixture was filtered, and the aqueous filtrate was adjusted to pH>8 by addition solid potassium bicarbonate. The mixture was then extracted with ethyl acetate (2×100 mL) and the combined organics were then concentrated before being subjected to column chromatography on silica gel using 40-70% ethyl acetate in cyclohexane to give 5-chloro-2-[[3-[chloro(difluoro)methyl]-1H-pyrazol-5-yl]methyl]pyrimidine 110 as an off-white solid (1.211 g). ¹H NMR (400 MHz, chloroform) δ=11.55 (br s, 1H), 8.70 (s, 2H), 6.49 (s, 1H), 4.40 (s, 2H)

### Step 6: Preparation of 2-[[2-butyl-5-[chloro(difluoro)methyl]pyrazol-3-yl]methyl]-5-chloro-pyrimidine (1.020)

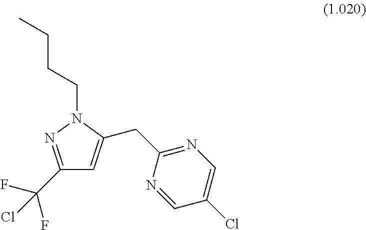 [SMILES: CCCCn1nc(C(F)(F)Cl)cc1Cc1ncc(Cl)cn1]

To a 25 mL flask was added 5-chloro-2-[[3-[chloro(difluoro)methyl]-1H-pyrazol-5-yl]methyl]pyrimidine 110 (106 mg, 0.380 mmol) and triphenylphosphine (200 mg, 0.763 mmol). The vessel was evacuated and backfilled with nitrogen gas three times, before 2-methyltetrahydrofuran (2.50 mL) and 1-butanol (0.070 mL, 0.76 mmol) were added and the resulting solution was stirred and cooled to 0 °C. Diisopropyl azodicarboxylate (0.140 mL, 0.711 mmol) was then added dropwise, and the resulting mixture was allowed to warm to room temperature and stirred for 1 hour. The reaction was quenched by addition of methanol (20 mL), and the resulting mixture was concentrated and subjected to column chromatography on silica gel using 0-30% ethyl acetate in cyclohexane. The product rich fractions were concentrated and subjected to reverse-phase column chromatography on C-18 silica gel using 60-100% acetonitrile in water, both with 0.1% formic acid to give 2-[[2-butyl-5-[chloro(difluoro)methyl]pyrazol-3-yl]methyl]-5-chloro-pyrimidine 1.020 as a colourless oil (21.1 mg). ¹H NMR (400 MHz, chloroform) δ=8.66 (s, 2H), 6.37 (s, 1H), 4.31 (s, 2H), 4.17 (t, 2H), 1.83-1.75 (m, 2H), 1.39-1.29 (m, 2H), 0.93 (t, 3H).

TABLE 1
Compounds of the present invention

| | | ¹HNMR (400 MHz, CDCl₃ unless |
|---|---|---|
| COMPOUND | STRUCTURE | otherwise stated) |
| 1.001 | 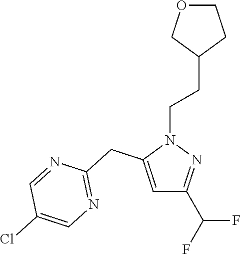 [SMILES: FC(F)c1cc(Cc2ncc(Cl)cn2)n(CCC2CCOC2)n1] | δ = 8.65 (s, 2H), 6.62 (t, 1H), 6.37 (s, 1H), 4.31 (s, 2H), 4.21- 4.13 (m, 2H), 3.90-3.81 (m, 2H), 3.78-3.70 (m, 1H), 3.35 (dd, 1H), 2.25-2.12 (m, 1H), 2.10-1.99 (m, 1H), 1.91 (q, 2H), 1.58-1.47 (m, 1H) |
| 1.002 | 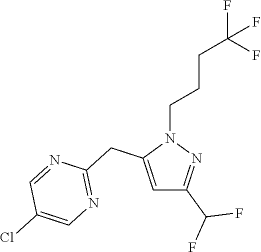 [SMILES: FC(F)c1cc(Cc2ncc(Cl)cn2)n(CCCC(F)(F)F)n1] | δ = 8.64 (s, 2H), 6.62 (t, 1H), 6.39 (s, 1H), 4.31 (s, 2H), 4.24 (t, 2H), 2.21-2.06 (m, 4H) |
| 1.003 | 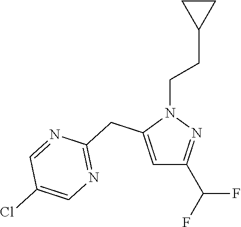 [SMILES: FC(F)c1cc(Cc2ncc(Cl)cn2)n(CCC2CC2)n1] | δ = 8.65 (s, 2H), 6.63 (t, 1H), 6.33 (s, 1H), 4.34 (s, 2H), 4.23 (t, 2H), 1.71 (q, 2H), 0.68- 0.57 (m, 1H), 0.46-0.40 (m, 2H), 0.05-−0.03 (m, 2H) |
| 1.004 | 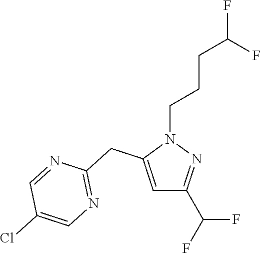 [SMILES: FC(F)CCCn1nc(C(F)F)cc1Cc1ncc(Cl)cn1] | δ = 8.64 (s, 2H), 6.62 (t, 1H), 6.38 (s, 1H), 5.82 (tt, 1H), 4.31 (s, 2H), 4.22 (t, 2H), 2.06-1.96 (m, 2H), 1.94-1.77 (m, 2H) |
| 1.005 | 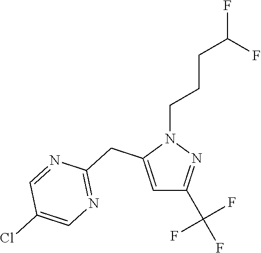 [SMILES: FC(F)CCCn1nc(C(F)(F)F)cc1Cc1ncc(Cl)cn1] | δ = 8.65 (s, 2H), 6.43 (s, 1H), 5.83 (tt, 1H), 4.32 (s, 2H), 4.26 (t, 2H), 2.10-1.96 (m, 2H), 1.94- 1.78 (m, 2H) |
| 1.006 | 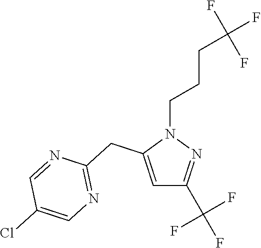 [SMILES: FC(F)(F)CCCn1nc(C(F)(F)F)cc1Cc1ncc(Cl)cn1] | δ = 8.64 (s, 2H), 6.43 (s, 1H), 4.32 (s, 2H), 4.30-4.24 (m, 2H), 2.20-2.08 (m, 4H) |
| 1.007 | 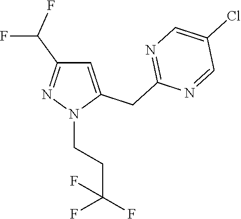 [SMILES: FC(F)c1cc(Cc2ncc(Cl)cn2)n(CCC(F)(F)F)n1] | 8.65 (s, 2 H), 6.62 (t, 1 H), 6.39 (s, 1 H), 4.41-4.47 (m, 2 H), 4.34 (s, 2 H), 2.73-2.84 (m, 2 H) |
| 1.008 | 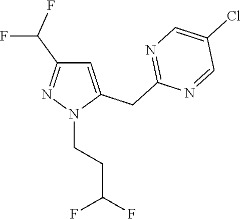 [SMILES: FC(F)CCn1nc(C(F)F)cc1Cc1ncc(Cl)cn1] | 8.65 (s, 2 H), 6.68 (t, 1 H), 6.40 (s, 1 H), 5.92 (tt, 1 H), 4.36 (t, 2H), 4.30 (s, 2 H), 2.41-2.48 (m, 2 H) |
| 1.009 | 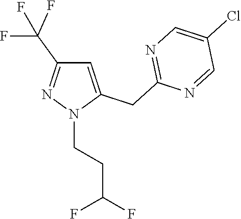 [SMILES: FC(F)CCn1nc(C(F)(F)F)cc1Cc1ncc(Cl)cn1] | |
| 1.010 | 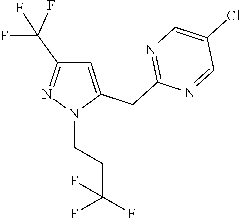 [SMILES: FC(F)(F)CCn1nc(C(F)(F)F)cc1Cc1ncc(Cl)cn1] | 8.66 (s, 2H), 6.44 (s, 1H), 4.44- 4.50 (m, 2 H), 4.35 (s, 2 H) 2.72- 2.89 (m, 3 H) 2.71-2.86 (m, 2 H) |
| 1.011 | 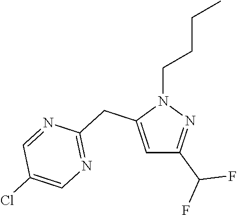 [SMILES: CCCCn1nc(C(F)F)cc1Cc1ncc(Cl)cn1] | 8.66 (s, 2 H), 6.64 (t, 1 H), 6.35 (s, 1 H), 4.32 (s, 2 H), 4.14 (t, 2 H), 1.74-1.82 (m, 2 H), 1.28- 1.39 (m, 2 H), 0.93 (t, 3 H) |
| 1.012 | 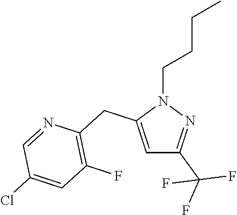 [SMILES: CCCCn1nc(C(F)(F)F)cc1Cc1ncc(Cl)cc1F] | |
| 1.013 | 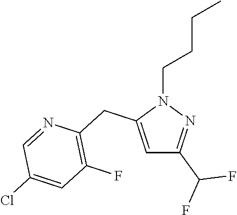 [SMILES: CCCCn1nc(C(F)F)cc1Cc1ncc(Cl)cc1F] | 8.36 (d, 1H), 7.47 (dd, 1H), 6.62 (t, 1H), 6.23 (s, 1H), 4.14-4.20 (m, 4H), 1.71-1.85 (m, 2H), 1.35 (dd, 2H), 0.94 (t, 3H) |
| 1.014 | 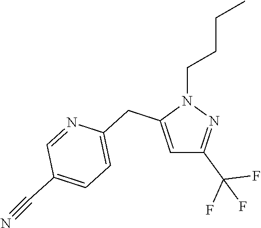 [SMILES: CCCCn1nc(C(F)(F)F)cc1Cc1ccc(C#N)cn1] | |
| 1.015 | 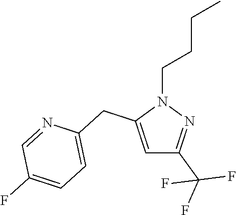 [SMILES: CCCCn1nc(C(F)(F)F)cc1Cc1ccc(F)cn1] | |
| 1.016 | 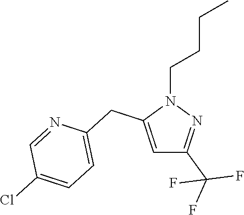 [SMILES: CCCCn1nc(C(F)(F)F)cc1Cc1ccc(Cl)cn1] | |
| 1.017 | 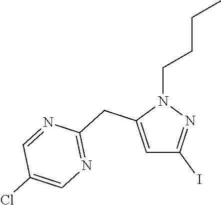 [SMILES: CCCCn1nc(I)cc1Cc1ncc(Cl)cn1] | |
| 1.018 | 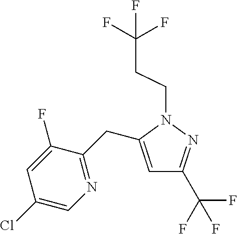 [SMILES: Fc1cc(Cl)cnc1Cc1cc(C(F)(F)F)nn1CCC(F)(F)F] | 8.35 (d, 1H), 7.49 (dd, 1H), 6.35 (s, 1H), 4.46-4.50 (m, 2H), 4.22 (d, 2H), 2.72-2.84 (m, 2H) |
| 1.019 | 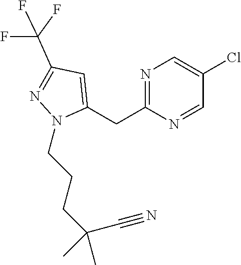 [SMILES: CC(C)(C#N)CCCn1nc(C(F)(F)F)cc1Cc1ncc(Cl)cn1] | 8.66 (s, 2H), 6.63 (t, 1 H), 6.39 (s, 1 H), 4.33 (s, 2 H), 4.21 (t, 2 H), 2.00-2.09 (m, 2H), 1.48- 1.56 (m, 2H) 1.33 (s, 6H) |
| 1.020 | 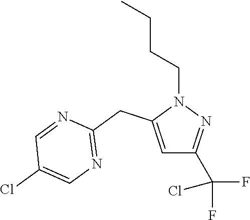 [SMILES: CCCCn1nc(C(F)(F)Cl)cc1Cc1ncc(Cl)cn1] | 8.66 (s, 2H), 6.37 (s, 1H), 4.31 (s, 2H), 4.17 (t, 2H), 1.75-1.83 (m, 2H), 1.29-1.39 (m, 2H), 0.93 (t, 3H) |
| 1.021 | 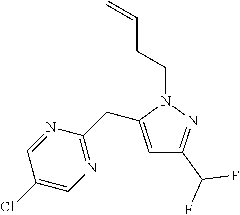 [SMILES: C=CCCn1nc(C(F)F)cc1Cc1ncc(Cl)cn1] | 8.65 (s, 2H), 6.64 (t, 1H), 6.36 (s, 1H), 5.68-5.76 (m, 1H), 4.99- 5.09 (m, 2H), 4.32 (s, 2H), 4.21 (t, 2H), 2.57 (q, 2H) |
| 1.022 | 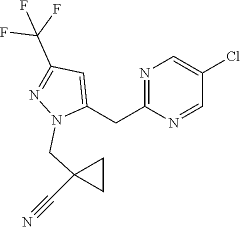 [SMILES: N#CC1(Cn2nc(C(F)(F)F)cc2Cc2ncc(Cl)cn2)CC1] | 8.66 (s, 2H), 6.49 (s, 1H), 4.50 (s, 2H), 4.45 (s, 2H), 1.25-1.42 (m, 4H) |
| 1.023 | 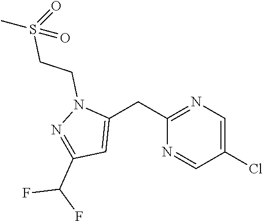 [SMILES: CS(=O)(=O)CCn1nc(C(F)F)cc1Cc1ncc(Cl)cn1] | 1H NMR (400 MHz, METHANOL-d4) δ ppm 8.80 (s, 2H), 6.72 (t, 1H), 6.40 (s, 1H), 4.66 (t, 2H), 4.51 (s, 2H), 3.77 (t, 2H), 2.83 (s, 3H). |
| 1.024 | 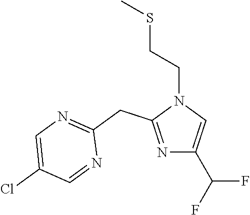 [SMILES: CSCCn1cc(C(F)F)nc1Cc1ncc(Cl)cn1] | 8.65 (s, 2 H), 6.63 (t, 1H), 6.38 (s, 1H), 4.39( s, 2H), 4.38 (t, 2H) 2.94 (t, 2H) 2.04 (s, 3H) |
| 1.025 | 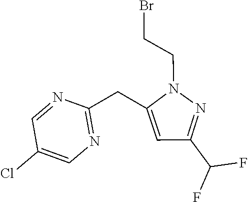 [SMILES: FC(F)c1cc(Cc2ncc(Cl)cn2)n(CCBr)n1] | 8.65 (s, 2H), 6.63 (t, 1H), 6.39 (s, 1H), 4.58 (t, 2H), 4.40 (s, 2H), 3.74 (t, 2H) |
| 1.026 | 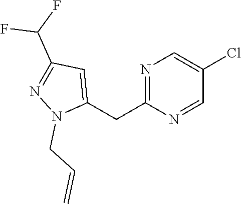 [SMILES: C=CCn1nc(C(F)F)cc1Cc1ncc(Cl)cn1] | 8.65 (s, 2H), 6.65 (t, 1H), 6.40 (s, 1H), 5.89-5.99 (m, 1H), 5.19 (dd, 1H), 5.01 (dd, 1H), 4.86 (dd, 2H), 4.31 (s, 2H) |
| 1.027 | 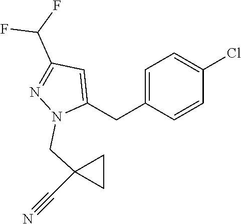 [SMILES: N#CC1(Cn2nc(C(F)F)cc2Cc2ccc(Cl)cc2)CC1] | 8.65 (s, 2H), 6.63 (t, 1H), 6.44 (s, 1H), 4.47 (s, 2H), 4.41 (s, 2H), 1.23-1.29 (m, 2H), 1.20- 1.24 (m, 2H) |
| 1.028 | 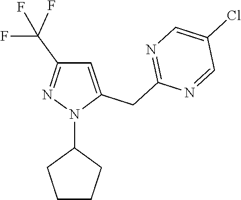 [SMILES: FC(F)(F)c1cc(Cc2ncc(Cl)cn2)n(C2CCCC2)n1] | 8.66 (s, 2H), 6.37 (s, 1H), 4.71- 4.79 (m, 1H), 4.35 (s, 2H), 1.90- 2.11 (m, 6H), 1.58-1.70 (m, 2H) |
| 1.029 | 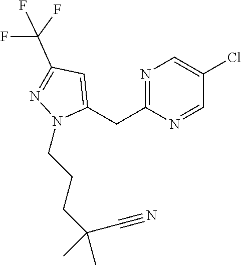 [SMILES: CC(C)(C#N)CCCn1nc(C(F)(F)F)cc1Cc1ncc(Cl)cn1] | 8.67 (s, 2H), 6.44 (s, 1H), 4.33 (s, 2H), 4.25 (t, 2H), 2.00-2.10 (m, 2H), 1.48-1.58 (m, 2H), 1.33 (s, 6H) |
| 1.030 | 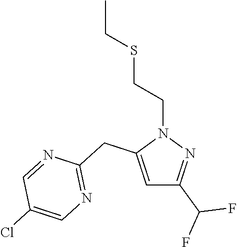 [SMILES: CCSCCn1nc(C(F)F)cc1Cc1ncc(Cl)cn1] | 8.65 (s, 2H), 6.63 (t, 1H), 6.37 (s, 1H), 4.34 (t, 2H), 4.39 (s, 2H), 2.97 (t, 2H), 2.42 (q, 2H), 1.23 (t, 3H) |
| 1.031 | 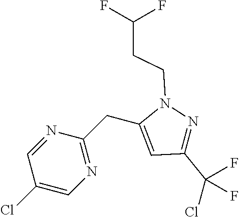 [SMILES: FC(F)CCn1nc(C(F)(F)Cl)cc1Cc1ncc(Cl)cn1] | 8.65 (s, 2H), 6.40 (s, 1H), 5.93 (tt, 1H), 4.38 (t, 2H), 4.33 (s, 2H), 2.41-2.55 (m, 2H) |
| 1.032 | 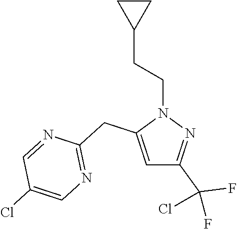 [SMILES: FC(F)(Cl)c1cc(Cc2ncc(Cl)cn2)n(CCC2CC2)n1] | 8.65 (s, 2H), 6.36 (s, 1H), 4.33 (s, 2H), 4.26 (t, 2H), 1.73 (q, 2H), 0.58-0.68 (m, 1H), 0.39- 0.46 (m, 2H), 0.00-0.03 (m, 2H) |
| 1.033 | 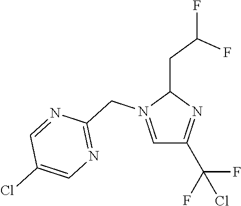 | 8.65 (s, 2H), 6.46 (s, 1H), 6.17 (tt, 1H), 4.66 (td, 2H), 4.36 (s, 2H) |
| 1.034 | 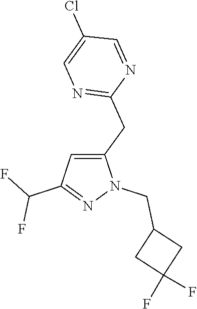 [SMILES: FC(F)c1cc(Cc2ncc(Cl)cn2)n(CC2CC(F)(F)C2)n1] | 8.66 (s, 2H), 6.61 (t, 1H), 6.38 (s, 1 H), 4.26-4.33 (m, 4H), 2.58-2.78 (m, 3H), 2.29-2.37 (m, 2H) |
| 1.035 | 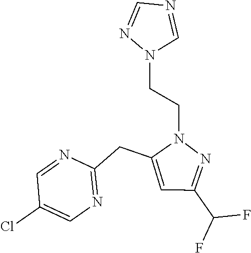 [SMILES: FC(F)c1cc(Cc2ncc(Cl)cn2)n(CCn2cncn2)n1] | 8.60 (s, 2 H), 7.94 (s, 1 H), 7.72 (s, 1 H), 6.64 (t, 1 H), 6.31 (s, 1 H), 4.75 (t, 2H), 4.64 (t, 2 H), 4.01 (s, 2 H) |
| 1.036 | 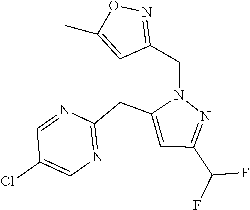 [SMILES: Cc1cc(Cn2nc(C(F)F)cc2Cc2ncc(Cl)cn2)no1] | 8.61 (s, 2 H), 6.64 (t, 1 H), 6.40 (s, 1 H), 5.86(s, 1 H), 5.48 (s, 2 H), 4.35 (s, 2H), 2.38 (s, 3 H) |
| 1.037 | 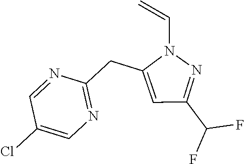 [SMILES: C=Cn1nc(C(F)F)cc1Cc1ncc(Cl)cn1] | 8.66 (s, 2 H), 7.14-7.20 (dd, 1 H), 6.67 (t, 1 H), 6.47 (s, 1 H), 5.83 (d,1 H), 4.98 (d,1 H), 4.38 (s, 2 H) |
| 1.038 | 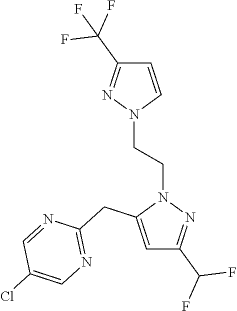 [SMILES: FC(F)c1cc(Cc2ncc(Cl)cn2)n(CCn2ccc(C(F)(F)F)n2)n1] | 8.59 (s, 2 H), 7.02 (br s, 1 H), 6.64 (t, 1 H), 6.41 (d, 1 H), 6.27 (s, 1 H), 4.63-4.72 (m, 4 H), 3.93 (s, 2 H) |
| 1.039 | 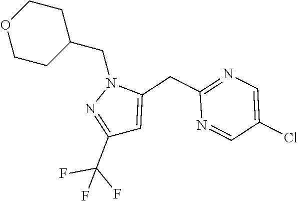 [SMILES: FC(F)(F)c1cc(Cc2ncc(Cl)cn2)n(CC2CCOCC2)n1] | 8.65 (s, 2 H) 6.39 (s, 1 H) 4.31 (s, 2 H) 4.05 (d, 2 H) 3.94 (dd, 2 H) 3.33 (td, 2 H) 2.21 (ddd, 1 H) 1.42-1.52 (m, 2 H) 1.29-1.40 (m, 2 H) |
| 1.040 | 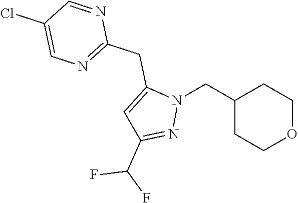 [SMILES: FC(F)c1cc(Cc2ncc(Cl)cn2)n(CC2CCOCC2)n1] | 8.65 (s, 2 H) 6.62 (s, 1 H) 6.35 (s, 1 H) 4.31 (s, 2 H) 3.93-4.05 (m, 4 H) 3.34 (td, 2 H) 2.16- 2.24 (m, 1 H) 1.44-1.54 (m, 2 H) 1.27-1.41 (m, 2 H) |
| 1.041 | 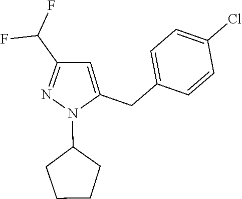 [SMILES: FC(F)c1cc(Cc2ccc(Cl)cc2)n(C2CCCC2)n1] | 8.65 (s, 2 H) 6.64 (t, 1 H) 6.34 (s, 1 H) 4.65-4.80 (m, 1 H) 4.34 (s, 2 H) 1.85-2.06 (m, 6 H) 1.57- 1.69 (m, 2 H) |
| 1.042 | 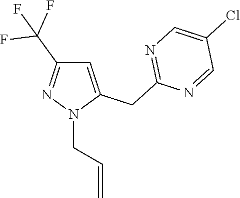 [SMILES: C=CCn1nc(C(F)(F)F)cc1Cc1ncc(Cl)cn1] | 8.63 (s, 2 H) 6.42 (s, 1 H) 5.86- 5.97 (m, 1 H) 5.17 (d, 1 H) 4.97 (d, 1 H) 4.86 (d, 2 H) 4.29 (s, 2 H) |
| 1.043 | 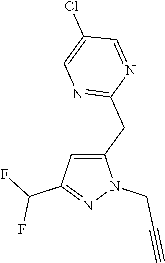 [SMILES: C#CCn1nc(C(F)F)cc1Cc1ncc(Cl)cn1] | 8.65 (s, 2 H) 6.64 (t, 1 H) 6.43 (s, 1 H) 5.09 (d, 2 H) 4.45 (s, 2 H) 2.40 (t, 1 H) |

### Biological Examples

Seeds of a variety of test species are sown in standard soil in pots Amaranthus palmeri (AMAPA), Amaranthus retoflexus (AMARE), Echinochloa crus-galli (ECHCG), Ipomoea hederacea (IPOHE), Setaria faberi (SETFA). After cultivation for one day (pre-emergence) or after 8 days cultivation (post-emergence) under controlled conditions in a glasshouse (at 24/16 °C, day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16 °C, day/night; 14 hours light; 65% humidity) and watered twice daily. After 13 days for pre and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| Com- | Rate | | | | | |
|---|---|---|---|---|---|---|
| pound | (g/ha) | AMARE | AMAPA | SETFA | ECHCG | IPOHE |
| 1.001 | 250 | 1 | 1 | NT | 1 | 2 |
| 1.002 | 250 | 2 | 1 | NT | 1 | 2 |
| 1.003 | 250 | 1 | 1 | NT | 1 | 3 |
| 1.004 | 250 | 3 | 3 | NT | 1 | 2 |
| 1.005 | 250 | 5 | NT | 4 | 4 | 3 |
| 1.006 | 250 | 5 | 5 | 4 | 3 | 3 |
| 1.007 | 250 | 4 | 3 | NT | 2 | 3 |
| 1.008 | 250 | 3 | 3 | 1 | 1 | 4 |
| 1.010 | 250 | 5 | 4 | NT | 4 | 3 |
| 1.011 | 250 | 2 | 2 | NT | 1 | 2 |
| 1.013 | 250 | 2 | 2 | 1 | 1 | 2 |
| 1.018 | 250 | 1 | 4 | 4 | 4 | 4 |
| 1.019 | 250 | 1 | 1 | 1 | 1 | 1 |
| 1.020 | 250 | 5 | 4 | 1 | 2 | 2 |
| 1.021 | 250 | 3 | 2 | 1 | 1 | 1 |
| 1.022 | 250 | 4 | 4 | 1 | 1 | 4 |
| 1.023 | 250 | 2 | 2 | 1 | 1 | 3 |
| 1.025 | 250 | 1 | 1 | 1 | 1 | 1 |
| 1.026 | 250 | 1 | 1 | 1 | 1 | 1 |
| 1.027 | 250 | 1 | 1 | 1 | 1 | 1 |
| 1.028 | 250 | 4 | 2 | 2 | 2 | 3 |
| 1.029 | 250 | 4 | 3 | 2 | 3 | 3 |
| 1.030 | 250 | 5 | 5 | 4 | 2 | 5 |
| 1.031 | 250 | 4 | 4 | 4 | 3 | 4 |
| 1.032 | 250 | 5 | 5 | 5 | 4 | 4 |
| 1.033 | 250 | 5 | 5 | 3 | 2 | 4 |
| 1.037 | 250 | 4 | 3 | 2 | 2 | 2 |
| 1.038 | 250 | 1 | 1 | 1 | 1 | 1 |

TABLE B2
Pre-emergence Test

| Com- | Rate | | | | | |
|---|---|---|---|---|---|---|
| pound | (g/ha) | AMARE | AMAPA | SETFA | ECHCG | IPOHE |
| 1.001 | 250 | 1 | 2 | NT | 1 | 2 |
| 1.002 | 250 | 5 | 5 | NT | 1 | 3 |
| 1.003 | 250 | 4 | 4 | NT | 1 | 1 |
| 1.004 | 250 | 4 | 4 | NT | 1 | 2 |
| 1.005 | 250 | 5 | 5 | 5 | 4 | 5 |
| 1.006 | 250 | 5 | 5 | 5 | 4 | 3 |
| 1.007 | 250 | 5 | 5 | NT | 2 | 4 |
| 1.008 | 250 | 5 | 5 | 4 | 4 | 3 |
| 1.010 | 250 | 5 | 5 | NT | 5 | 5 |
| 1.011 | 250 | 5 | 5 | NT | 2 | 3 |
| 1.013 | 250 | 4 | 3 | 3 | 3 | 2 |
| 1.018 | 250 | 5 | 5 | 5 | 5 | 3 |
| 1.019 | 250 | 1 | 1 | 1 | 1 | 1 |
| 1.020 | 250 | 5 | 5 | 4 | 4 | 2 |
| 1.021 | 250 | 5 | 5 | 3 | 2 | 1 |
| 1.022 | 250 | 5 | 4 | 4 | 3 | 5 |
| 1.023 | 250 | 1 | 1 | 1 | 1 | 2 |
| 1.025 | 250 | 4 | 4 | 1 | 2 | 1 |
| 1.026 | 250 | 1 | 1 | 1 | 1 | 1 |
| 1.027 | 250 | 1 | 1 | 2 | 1 | 2 |
| 1.028 | 250 | 5 | 5 | 5 | 2 | 1 |
| 1.029 | 250 | 5 | 4 | 3 | 3 | 5 |
| 1.030 | 250 | 1 | 1 | 1 | 1 | 1 |
| 1.031 | 250 | 5 | 5 | 5 | 5 | 5 |
| 1.032 | 250 | 5 | 5 | 5 | 5 | 5 |
| 1.033 | 250 | 5 | 5 | 5 | 5 | 5 |
| 1.037 | 250 | 5 | 5 | 1 | 1 | 3 |
| 1.038 | 250 | 1 | 2 | 2 | 2 | 1 |

NT = Not tested.

1. A compound of Formula (I): 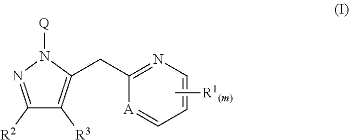
or an agronomically acceptable salt thereof,
wherein
A is CR¹¹ or N;
Q is selected from the group consisting of hydrogen, C₁-C₈ alkyl, C₂-C₈ alkenyl, C₂-C₈ alkynyl, C₁-C₈ haloalkyl, C₂-C₈ haloalkenyl, C₂-C₈ haloalkynyl, C₁-C₄alkoxy-C₁-C₃alkyl-, C₁-C₄haloalkoxy-C₁-C₃alkyl-, C₁-C₄alkoxy-C₁-C₃haloalkyl-, C₁-C₈cyanoalkyl-, C₃-C₆ cycloalkyl (optionally substituted by CN, fluoro or chloro), —S(O)ₚR⁴ and —(CH₂)ₙR¹⁰;
R¹ is independently selected from the group consisting of halogen, —CN, C₁-C₂alkyl, C₁-C₂haloalkyl, C₃-C₆cycloalkyl, C₁-C₂alkoxy- and C₁-C₂haloalkoxy-;
R² is selected from the group consisting of halogen, —CN, NO₂, C₁-C₄alkyl, C₁-C₄haloalkyl, C₁-C₄alkoxy, —C(O)C₁-C₄alkyl, —C(O)OC₁-C₄alkyl, C₁-C₄haloalkoxy, —S(O)ₚC₁-C₄alkyl, —C(R⁶)═NOR⁷ and C₃-C₆cycloalkyl;
R³ is selected from the group consisting of hydrogen, halogen, C₁-C₄ alkyl, C₁-C₄ haloalkyl, C₁-C₄ alkoxy, C₁-C₄ haloalkoxy, —CN, NO₂, C₂-C₄alkenyl, C₂-C₄alkynyl, —S(O)ₚC₁-C₄alkyl, —S(O)ₚC₁-C₄haloalkyl, —C(O)OC₁-C₄alkyl and —C(O)NR⁸R⁹;
R⁴ is selected from the group consisting of C₁-C₄alkyl, C₁-C₄haloalkyl and C₃-C₆cycloalkyl;
R⁵ is selected from the group consisting of hydrogen, methyl, ethyl, —(CH₂)-cyclopropyl, C₂-C₃ alkenyl, C₂-C₃ alkynyl and cyclopropyl;
R⁶ is selected from the group consisting of hydrogen, methyl and ethyl;
R⁷ is methyl or ethyl;
R⁸ is hydrogen or C₁-C₄alkyl;
R⁹ is hydrogen or C₁-C₄alkyl;
R¹⁰ is selected from the group consisting of —OH, —C(H)O, —C(O)R⁴, —S(O)ₚR⁴, —C(H)(OH)—CH₂OH, —C(R⁶)═NOR⁵, —NR¹²R¹³, C₃-C₆ cycloalkyl (optionally substituted by CN, fluoro or hydroxy), a C₃-C₆ saturated heterocycle comprising one or more oxygen atoms, and a 5-membered heteroaryl which comprises from 1 to 3 heteroatoms each independently selected from the group consisting of oxygen, nitrogen and sulphur, wherein said heteroaryl is optionally substituted by one or two substituents independently selected from the group consisting of halogen, cyano, C₁-C₂ alkyl, C₁-C₂ haloalkyl, C₃-C₄cycloalkyl, C₁-C₂ alkoxy and C₁-C₄ haloalkoxy;
R¹¹ is selected from the group consisting of hydrogen, fluoro, chloro and CN;
R¹² and R¹³ are independently selected from the group consisting of C₁-C₄alkyl, C₃-C₄cycloalkyl, C₁-C₄alkyl-S(O)ₚ—, C₃-C₄cycloalkyl-S(O)ₚ—, C₁-C₄alkyl-C(O)— and C₃-C₄cycloalkyl-C(O)—;
m=0, 1 or 2;
n=1, 2 or 3; and
p=0, 1 or 2.

2. A compound of Formula (I) according to claim 1, which is of Formula (Ia) 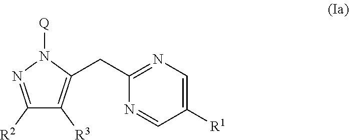
wherein Q, R¹, R² and R³ are as defined in claim 1 above.

3. A compound according to claim 1, wherein R¹ is chloro.

4. A compound according to claim 1, wherein R² is halogen or C₁-C₄haloalkyl.

5. A compound according to claim 1, wherein R³ is hydrogen or halogen.

6. A compound according to claim 1, wherein Q is selected from the group consisting of hydrogen, C₁-C₈ alkyl, C₂-C₈ alkenyl, C₁-C₈ haloalkyl, C₁-C₄alkoxy-C₁-C₃alkyl-, C₁-C₄haloalkoxy-C₁-C₃alkyl-, —S(O)ₚR⁴ and —(CH₂)ₙR¹⁰.

7. A compound according to claim 6, wherein Q is —(CH₂)ₙR¹⁰, n is 1, 2 or 3 and R¹⁰ is selected from the group consisting of —OH, —C(H)(OH)—CH₂OH and —C(H)═NOR⁵.

8. A compound according to claim 6, wherein Q is C₁-C₈ alkyl.

9. A compound according to claim 6, wherein Q is C₁-C₈ haloalkyl.

10. A compound according to claim 6, wherein Q is C₁-C₄haloalkoxy-C₁-C₃alkyl-.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling weeds at a locus comprising application to the locus of a weed controlling amount of a composition according to claim 11.

15. A compound of Formula (II-a): 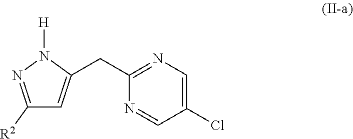
wherein R² is CF₂H or CF₂Cl.

16. Use of a compound of Formula (I) as defined in claim 1 as a herbicide.
